(19) United States
(12) Patent Application Publication
(10) Pub. No.: US 2006/0218669 A1
(43) Pub. Date: Sep. 28, 2006

(54) Sialylation of glycoproteins in plants

(76) Inventors: Lokesh Joshi, Temple, AZ (US); Miti M. Shah, Scottsdale, AZ (US)

Correspondence Address: NEEDLE & ROSENBERG, P.C., SUITE 1000, 999 PEACHTREE STREET, ATLANTA, GA, 30309-3915, US

(21) Appl. No.: 10/545,252

(22) PCT Filed: Feb. 11, 2004
(86) PCT No.: PCT/US04/04281

§ 371 Date: Feb. 27, 2006

Related U.S. Application Data
(60) Provisional application No. 60/446,477, filed on Feb. 11, 2003.

(51) Int. Cl. A01H 1/00; C07K 14/415; C12Q 1/68; C12N 15/82; C12N 5/04; A01H 5/00

(52) U.S. Cl. 800/284; 435/419; 435/468; 530/370; 435/6; 800/317.3

(57) ABSTRACT

The presence of sialic acids (SAs) at the non-reducing terminal of various glycoconjugates plays important roles in the function and half-life of glycoproteins in mammals and many species of microorganisms. It has been previously unknown that plant cells have the capacity to sialylate glycoproteins. The present invention discloses the presence of sialylated glycoconjugates and identifies N-acetylneuranminic acid (Neu5Ac) and N-glycolylneuraminic (Neu5Gc) on glycoproteins in suspension-cultured cells of plants, for example, Arabidopsis thaliana (A. thaliana), Nicotiana tabacum (tobacco), and Medicago sativa (alfalfa), extending the complexity of glycan structures known in the plant kingdom. This invention further discloses methods for engineering plants to produce recombinant glcyoproteins.

### CLAIM TO DOMESTIC PRIORITY

This application claims benefit of priority to U.S. provisional application Ser. No. 60/446,477, entitled “Mammalian-Like Sialyated Glycoproteins in Plants”, filed Feb. 11, 2003, by Joshi et al., and which is herein incorporated by reference in its entirety.

### FIELD OF THE INVENTION

This invention concerns generally glycoproteins in animals and plants, and more specifically the sialylated glycoconjugates in plants and methods for engineering plants to produce recombinant glycoconjugates.

### BACKGROUND OF THE INVENTION

Glycosylation is one of the most frequently occurring and important post-translational modifications. Most cell surface and secreted proteins are glycosylated in endoplasmic reticulum (ER) and Golgi by covalent attachment of sugar residues to asparagine (N-glycans) or to serine/threonine (O-glycans) side chains of the proteins (Varki, 1999a and b) (see also, FIG. 1). In vertebrates, sialic acid SA residues occupy the non-reducing terminal of most oligosaccharide chains on cell suface bound and secreted glycoproteins and glycolipids. There are increasing numbers of reports showing the presence of SAs in bacterial polysaccharides, Drosophila eggs, insect cell lines, fungi and now plant cells (Schauer, 2000; Joshi et al., 2001; Shah et al., 2003).

In mammals, the non-reducing terminal SA is essential, among other functions, for intermolecular communication and extending the half-life of glycoconjugates in circulation. Glycoproteins lacking SA on their glycan chains are recognized as “foreign” by asialoglycoprotein receptors and immune cells, removed from the serum and destroyed (Ashwell and Morell, 1974).

SAs are a diverse family of nine-carbon keto-sugar acids (FIG. 2) (Varki, 1992; Yasuo, 1999). More than 40 different species of SAs are found in nature, and the most common is N-acetylneuraminic acid (Neu5Ac), followed by N-glycolylneuraminic acid (Neu5Gc). Neu5Ac is the primary SA and most other forms of SAs are metabolically derived from it by hydroxylation (glycosylation), O-acetylation, lactylation, methylation, sulfation or phosphorylation (Varki, 1992). Therefore, according to this disclosure, the terms Neu5Ac and SA are used interchangeably. For example, Neu5Gc is synthesized from CMP-Neu5Ac in the cytoplasm by CMP-Neu5Ac hydroxylase, which is an iron dependent enzyme that utilizes the common electron transport chain of cytochrome b₅ and b₅ reductase (Shaw and Schauer, 1981; Gollub et al., 1998).

The carboxyl group at the 1-carbon position of SA is typically ionized at physiological pH, giving it a negative charge. SAs can form different α-linkages from its anomeric carbon to the 3- or 6-position of galactose residues or the 6-position of GalNAc residues. The anomeric carbon-2 of SA also can form linkage to the 8-position of another SA, yielding polysialic acid structures as in colominic acids and neural cell adhesion molecules (N-CAMs). Combination of all these properties gives structural and functional diversity to Sas, which can present themselves in multiple ways on glycoproteins and glycolipids.

In animals, SA metabolism can be divided into three distinct processes: first, synthesis of Neu5Ac in the cytoplasm and its activation to CMP-Neu5Ac in the nucleus; second, transfer of Neu5Ac to the appropriate oligosaccharide acceptor (FIG. 3); and third, removal and degradation of Neu5Ac, primarily in the lysosome (Reutter et al., 1997).

The de novo synthesis of CMP-Neu5Ac is the result of a complex pathway that involves multiple steps in the cytosol beginning with glucose (FIG. 3, Keppler et al., 1999). One of the central intermediates in this pathway is UDP-N-acetyl-D-glucosamine (UDP-GlcNAc), which is epimerized to N-acetyl-D-mannosamine (ManNAc) by UDP-GlcNAc-2-epimerase (Comb and Roseman, 1960). ManNAc is then phosphorylated by ManNAc-6-kinase to form ManNAc-6-P. In animals, UDP-GlcNAc-2-epimerase and ManNAc-6-kinase activities are found in a single bifunctional enzyme that is highly conserved and assembles as a hexamer (Hinderlich et al., 1997; Effertz et al., 1999), and the reaction it catalyzes is considered as the first committed step toward SA biosynthesis. UDP-GlcNAc-2-epimerase is allosterically inhibited in a feedback mechanism by CMP-Neu5Ac (Reutter et al., 1997).

Several lines of evidence suggest that UDP-GlcNAc-2-epimerase/ManNAc-6-kinase catalyze the rate limiting steps in the formation of SA: a) mutations in the epimerase domain of the GNE gene (Hong and Stanley, 2003) or epigenetically mediated loss of the enzyme expression (Oetke et al., 2003) result in a hyposialylated phenotype; b) the overall sialylation of cell-surface glycoconjugates of the hyposialylated cell lines increases when the medium is supplemented with ManNAc or D-mannosamine, but not with GlcNAc, D-glucosamine, D-mannose or D-glucose (Keppler et al., 1999).

In animal cells, Neu5Ac-9-phosphate synthase condenses phosphoenolpyruvate (PEP) with ManNAc-6-P to produce N-acetyl-D-neuraminic acid-9-phosphate (Neu5Ac-9-P). Neu5Ac-9-P is then dephosphorylated by Neu5Ac-9-P phosphatase to form Neu5Ac (Lawrence et al., 2000). The Escherichia coli SA synthase gene (neuB), which has been cloned and characterized, encodes an enzyme that directly converts phosphoenolpyruvate (PEP) and ManNAc to Neu5Ac (Vann et al., 1997). Thus in prokaryotes, the pathway is different in that SA synthase condenses PEP with ManNAc to form Neu5Ac (SA) without any phosphorylation and dephosphorylation steps (Vann et al., 1997; Ringenberg et al., 2003).

SA synthase gene from Drosophila melanogaster and human cDNA libraries have also been identified based on their high homology to E. coli SA synthase (neuB) (Kim et al., 2002). It is unclear whether Neu5Ac-9-phosphate synthase and SA synthase are significantly different enzymes, therefore, according to this disclosure, the names of these two enzymes will be used interchangeably.

Neu5Ac made in cytoplasm is then activated by CTP to form nucleotide sugar CMP-Neu5Ac (CMP-SA) in the nucleus; a reaction catalyzed by the CMP-Neu5Ac synthetase (Corfield et al., 1979). Finally, CMP-Neu5Ac is transported to the Golgi apparatus and pumped across its membranes by the action of a specific antiporter, CMP-Neu5Ac (CMP-SA) transporter. Transport of the nucleotide-sugars to the appropriate compartment of ER or Golgi apparatus is the prerequisite to a successful glycosylation reaction. Nucleotide-sugar transporters are Golgi membrane resident hydrophobic proteins that exist as functional dimers (Gerardy-Schahn et al., 2001).

In animals, CMP-SA transporter facilitates the transport of CMP-SA to the Golgi lumen in a non-energy dependent fashion. Mammalian cells lacking CMP-SA transporter make incomplete sugar chains, suggesting that glycosylation may be partially controlled by regulating the transporter and thereby regulating the amount of nucleotide sugar available in the Golgi (Oelmann et al., 2001).

Once CMP-SA is transported into the Golgi lumen, the transfer of SA onto oligosaccharide chains bound to proteins and lipids is facilitated by a large family of substrate-specific sialyltransferases (STs). The transfer of SAs on glycoprotein linked oligosaccharide chains is catalyzed by STs of alpha-2,3 sialyltransferase (ST3Gal), alpha-2,6 sialyltransferase (ST6Gal) and alpha-2,8 sialyltransferase (ST8SA) families that are linkage specific to: α-2,3 (NeuAcα-2,3Galα-1,4GlcNAc or NeuAcα-2,3Galα-1,3GalNAc-O-Ser/Thr), α-2,6 (NeuAcα-2,6Galα-1,4GlcNAc or NeuAcα-2,6GalNAc-O-Ser/Thr), and α-2,8 (NeuAcα-2,8NeuAc) positions, respectively. In animals, the expression of STs is species, organ, tissue and cellular physiology dependent (Taniguchi et al., 2002).

STs are type II membrane proteins with a short NH₂-terminal cytoplasmic tail, 16-20 amino acid signal anchor, a highly variable stem region (20-100 amino acids) and a large COOH-terminal catalytic domain that is localized in the Golgi lumen (Paulson and Colley, 1989). The amino acid sequences of STs show sequence homology in three consensus sequence regions called sialylmotif L (long), sialylmotif S (short), and sialylmotif VS (very short) (Tsuji et al., 1996). Sialylmotif L is involved in the binding of the sugar donor CMP-SA while the sialylmotif S binds to both the donor and the acceptor molecules (Datta et al., 1998). Although the precise function of sialylmotif VS has not been determined, it has been suggested that it may be involved in the catalytic process (Geremia et al., 1997).

In vertebrates, sialic acids (SAs) are the most important and structurally diverse family of charged sugar residues involved in many biological and pathological interactions (Schauer, 1985; Kelm and Schauer, 1997). However, it has been as widely accepted that plants do not possess pathways for the biosynthesis of SA and its incorporation into glycoconjugates (Schauer, 2000).

### SUMMARY OF THE INVENTION

The following methods are useful for evaluation of the pathway of SA biosynthesis and its transfer to the oligosaccharide acceptors and glycoproteins in plants. Any number of plants may be used including, but not limited to, Arapidopsis thaliana, Nicotiana tabacum (tobacco) and Medicago satiwa (alfalfa).

A method for determining the influence of an enzyme on the sialic acid pathway in a plant is disclosed comprising carrying out an in vitro assay, wherein the assay is capable of determining the enzyme activities in an untransformed plant and constitutively over-expressing a gene that codes for the enzyme, wherein an effect of over-expression of the gene is capable of being quantified. The enzyme can be mammalian or prokaryotic and may include, but is not limited to UDP-GlcNAc-2-epimerase, ManNAc-6-kinase and sialic acid synthase.

Also disclosed is a method for functionally characterizing a CMP-sialic acid transporter in a plant comprising constitutively over-expressing a plant CMP-sialic acid transporter gene and quantifying an effect of over-expressing the plant CMP-sialic acid transporter gene. The level of sialic acid in a plant can be altered be up-regulating or down-regulating the CMP-sialic acid transporter transcript levels in the plant.

Further disclosed is a method for functionally characterizing a sialyltransferase in a plant, comprising constitutively over-expressing a plant sialyltransferase gene and quantifying an effect of over-expressing the plant sialyltransferase gene. The level of sialic acid in a plant can be altered be up-regulating or down-regulating the sialyltransferase transcript levels in the plant.

Finally, additional features of the disclosed methods are described in detail below and in the appended claims.

### BRIEF DESCRIPTION OF THE DRAWINGS

FIG. 1 shows the common sugar linkages in mammals;

FIG. 2 shows the parent sialic acid molecule known as Neuraminic acid;

FIG. 3 illustrates sialic acid biosynthesis and transfer to glycoconjugates;

FIGS. 4A and 4B illustrate the specificity of lectin binding to sialylated glycoproteins, specifically biotinylated SNA and MAA binding to A. thaliana suspension cultured cell total proteins.

FIGS. 5A and 5B show mass spectrometry analysis demonstrating the presence of Neu5Ac and Neu5Gc on A. thaliana glycoproteins;

FIGS. 6A, 6B and 6C show LC-ESI and MALDI-70F analysis results demonstrating the presence of Neu5Ac (FIG. 6B) and Neu5Gc (FIG. 6C);

FIG. 7 illustrates the topology and amino acid sequence alignment of selected CMP-SA transporters;

FIG. 8 shows the conserved amino acid motifs and alignment of sialyltransferase genes from A. thaliana and other organisms;

FIG. 9 is a MALDI MS spectra of standard and plant cell derived DMB-Neu5Ac (FIG. 9A) and DMB-Neu5Gc (FIG. 9B) illustrating the presence of Neu5Ac and Neu5Gc on A. thaliana glycoproteins (FIG. 9C) according to an alternate embodiment of the disclosure; and

FIG. 10 is the immunofluorescence microscopic images of plant cells to study localization and distribution of sialylated glycocongates with SNA-I: Smbucus nigra agglutinin-I (FIG. 10A), MAA: Maackia amurensis agglutinin (FIG. 10B), and TTM: Tritrichomonas mobilensis lectin (FIG. 10C).

### DETAILED DESCRIPTION

The present invention discloses plants that produce sialylated glycoconjugates containing both N-acetyl-D-neuraminic acid (Neu5Ac) and N-glycolyl-D-neuraninic acid (Neu5Gc). Our discovery of sialylated glycoconjugates in plants establishes a new paradigm in glyobiology, plant biochemistry and proteomics, and opens a new field of research in plant biochemistry and raises important questions regarding the biosynthesis, regulation, distribution, and function of SAs in plants. The discovery of the presence of SA structures in Arabidopsis thaliana (A. thaliana) and the recognition that biosynthetic pathways are relatively evolutionarily conserved among animals and bacteria led to an identification of the key enzymes required for plant SA biosynthesis.

Thus, the following methods are useful for evaluation of the pathway of SA biosynthesis and its transfer to the oligosaccharide acceptors and glycoproteins in plants. According to one embodiment of the disclosure, the plants are selected from a group comprising Arapidopsis thaliana, Nicotiana tabacum (tobacco) and Medicago satiwa (alfalfa).

Determination of the Influence of Mammalian UDP-GlcNAc-2-Epimerase/ManNAc-6-Kinase on the SA Biosynthetic Pathway in Plants.

In mammals, the bifunctional UDP-GlcNAc-2-epimerase/ManNAc-6-kinase is a key enzyme in SA metabolism; It controls the rate-limiting step in SA biosynthetic pathway and therefore is of importance for the regulation. In bacteria, an enzyme, SA lyase, is coordinately up-regulated with an increase in SA levels. Enzymes with UDP-GlcNAc-2-epimerase/ManNAc-6-kinase-like functions and significance exist in A. thaliana. However, extensive homology searches in the database have not identified a candidate A. thaliana UDP-GlcNAc-2-epimerase/ManNAc-6-kinase gene with high sequence homology to the mammalian or bacterial ones.

Thus, the following method is useful to evaluate the key role of ManNAc-6-P in plant SA biosynthesis: (1) carry out in vitro assays to determine UDP-GlcNAc-2-epimerase/ManNAc-6-kinase activities in untransformed plants; (2) constitutively over-express the mammalian UDP-GlcNAc-2-epimerase/ManNAc-6-kinase gene and quantify its impact on SA biosynthesis by measuring free and bound SA; and (3) determine SA lyase activity in transformed and untransformed plants. This method is also useful in providing insight into the UDP-GlcNAc-2-epimerase regulation in plant cells.

Determination of the Influence of Mammalian Neu5Ac Phosphate Synthase (SA Synthase) on the SA Biosynthetic Pathway in Plants.

Neu5Ac phosphate synthase (SA synthase) is a cytoplasmic enzyme that acts downstream of UDP-GlcNAc-2-epimerase/ManNAc kinase in the biosynthesis of Neu5Ac (SA). In mammals, it catalyzes the synthesis of Neu5Ac-9-P from ManNAc-6-P and phosphoenolpyruvate (PEP). However, in bacteria, SA synthase condenses ManNAc with PEP to form Neu5Ac.

Because it regulates the flux of ManNAc-6-P/ManNAc entering the SA biosynthesis and functions in both bacteria and mammals, SA synthase is of importance in SA metabolism. Once again, extensive homology searches and A. thaliana database have not identified a candidate A. thaliana gene with high sequence homology.

Thus, the following method is useful to evaluate the involvement of NeuAc-9-P as an intermediate in plant SA biosynthesis: (1) carryout in vitro assays to determine SA synthase activity in untransformed plants; and (2) constitutively over-express in plants the mammalian SA synthase gene and quantify its effect on plant sialylation by measuring the reaction product (Neu5Ac-9-P or Neu5Ac) and bound SA.

Functionally Characterize A. thaliana CMP-Sialic Acid Transporters.

In animals, CMP-SA transporter facilitates the transport of CMP-SA to the lumen of the Golgi apparatus. The A. thaliana genome encodes two proteins with high sequence homology to the mammalian and bacterial CMP-SA transporters. The following method is useful for determining that plant orthologs function to facilitate the transport of CMP-SA to the lumen of the Golgi apparatus: (1) constitutively over-express in plants the putative CMP-SA transporter genes; (2) down-regulate endogenous CMP-SA transporter transcript levels in plants using techniques such as post-transcriptional gene silencing (RNAi); (3) characterize CMP-SA transporter T-DNA mutants of; and (4) determine the levels of sialylation in untransformed, CMP-SA transporter-up-regulated, and CMP-SA transporter-down-regulated plants using molecular and biochemical approaches.

Functionally Characterize A. thaliana Sialyltransferases (STs).

In mammals, sialyltransferases (STs) transfer SA residues from CMP-SA to the terminal position on asialo-oligosaccharides bound to proteins and lipids. Homology searches in the A. thaliana genome database have identified three candidate genes for plant STs. Therefore, the following method is useful for determining that the sequence orthologs of mammalian STs in the A. thaliana genome encode enzymes that catalyze similar reactions: (1) constitutively over-express selected putative ST genes in the plants; (2) down-regulate endogenous ST transcript levels in plants using techniques such as post-transcriptional gene silencing (RNAi); (3) characterize ST T-DNA mutants of the plants; (4) determine the levels of sialylation of endogenous total proteins in untransformed, ST-up-regulated, and ST-down-regulated plants using molecular and biochemical approaches; and (5) express plant STs in E. coli and perform biochemical characterization.

Experimental Method for Determining Presence of SA.

1. Detection of Sialylated Glycoproteins in A. thaliana Cells by Lectin Binding.

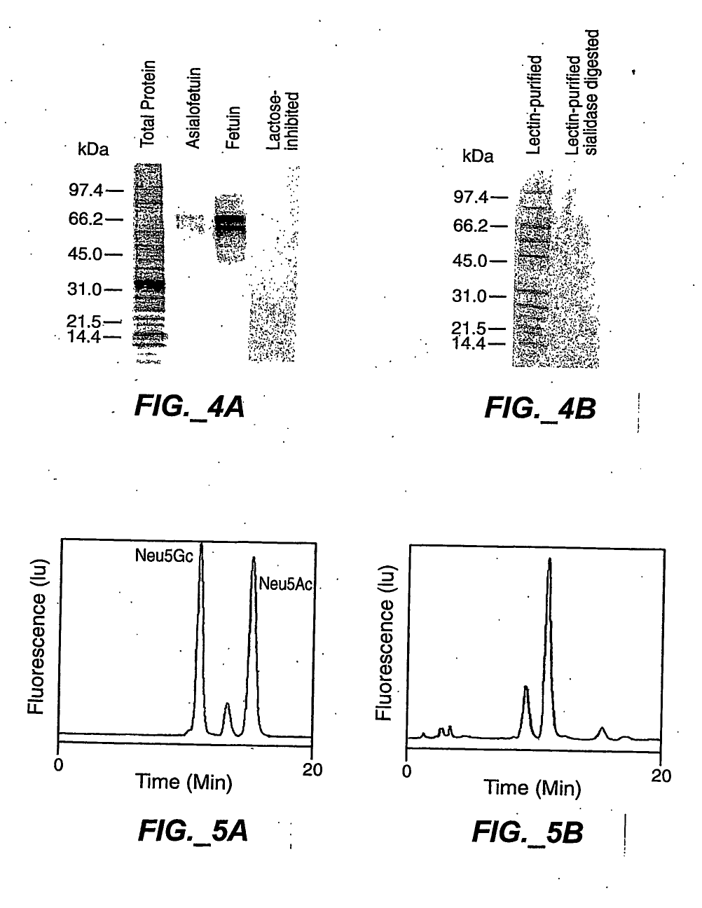

Glycoproteins from A. thaliana suspension-cultured cells grown on inorganic salts and sucrose were probed with a mixture of biotinylated SNA (Sambucus nigra) and MAA (Maackia amurensis) lectins, revealing terminal SAa-2,6-Gal and SAα-2,3-Gal structures (Shibuya et al., 1987; Wang and Cummings, 1988). As illustrated in FIG. 4, specificity of lectin binding to sialylated glycoproteins was verified by using fetuin, asialofetuin and inhibition of lectin binding by 100 mM lactose. FIG. 4 shows biotinylated SNA and MAA binding to A. thaliana suspension cultured cell total proteins. Positions of molecular weight standards are indicated to the left.

In FIG. 4A, total cell proteins from A. thaliana were resolved on 4-20% SDS/PAGE and transferred on PVDF membranes. Sialylated glycoproteins were detected colorimetrically (Sigmafast NBT/BCIP tablets) by binding of biotinylated SNA and MAA and avidin-alkaline phosphatase (Vector labs, CA) probes with 100 mM lactose, which inhibited binding of lectins to A. thaliana glycoproteins.

To further confirm these results, in FIG. 4B, sialylated glycoproteins were affinity-purified through immobilized SNA and MAA columns, digested with α-2-3,6 sialidase from Clostridium perfringens and probed with SNA and MAA lectins. The affinity-purified and sialidase-digested proteins did not bind to the lectins, confirming removal of α-2-3,6 linked SAs from A. thaliana glycoproteins.

2. Purification and Separation of SA by RP-HPLC and Mass Spectrometry.

SAs from A. thaliana proteins were released by mild acid hydrolysis (2 M acetic acid, 80° C. for 3 hr), purified through ion exchange chromatography and labeled with the fluorescent dye 1,2-diamino-4,5-methylene dioxybenzene (DMB, λₑₓ 373 nm, λₑₘ 448 nm). DMB-SA derivatives were separated on a reverse-phase C18 column on an Agilent 1100 workstation under isocratic conditions of 9% acetonitrile, 7% methanol and 84% water at flow rate of 0.65 ml/min (Hara et al., 1987).

In FIG. 5A, commercially available Neu5Ac and Neu5Gc were used as standard SAs. As shown by FIG. 5B, in A. thaliana samples, a prominent peak corresponding to Neu5Gc and a smaller peak corresponding to Neu5Ac were detected, indicating the presence of these two SAs on A. thaliana glycoconjugates. Similar results were observed when SAs were removed from glycoconjugates by α-2-3,6 sialidase treatment.

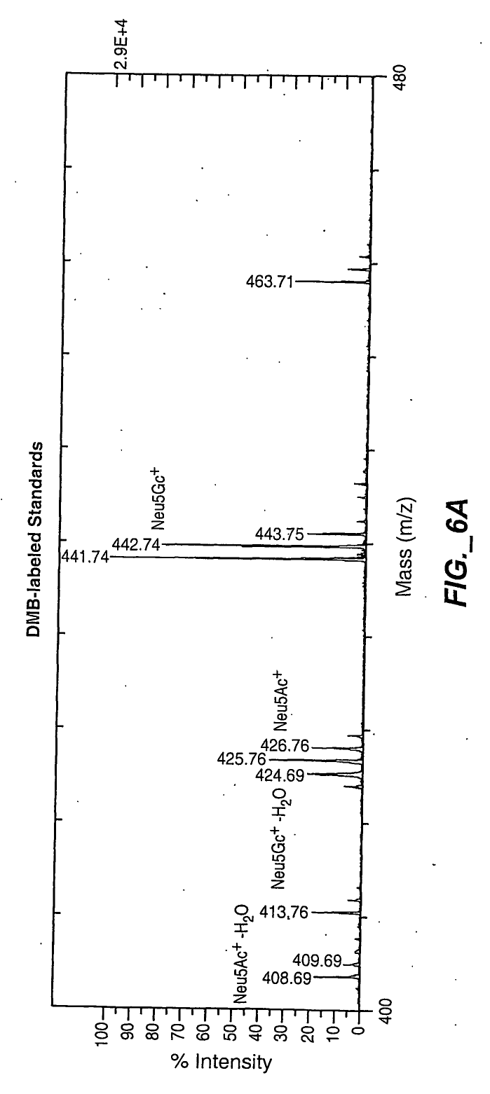

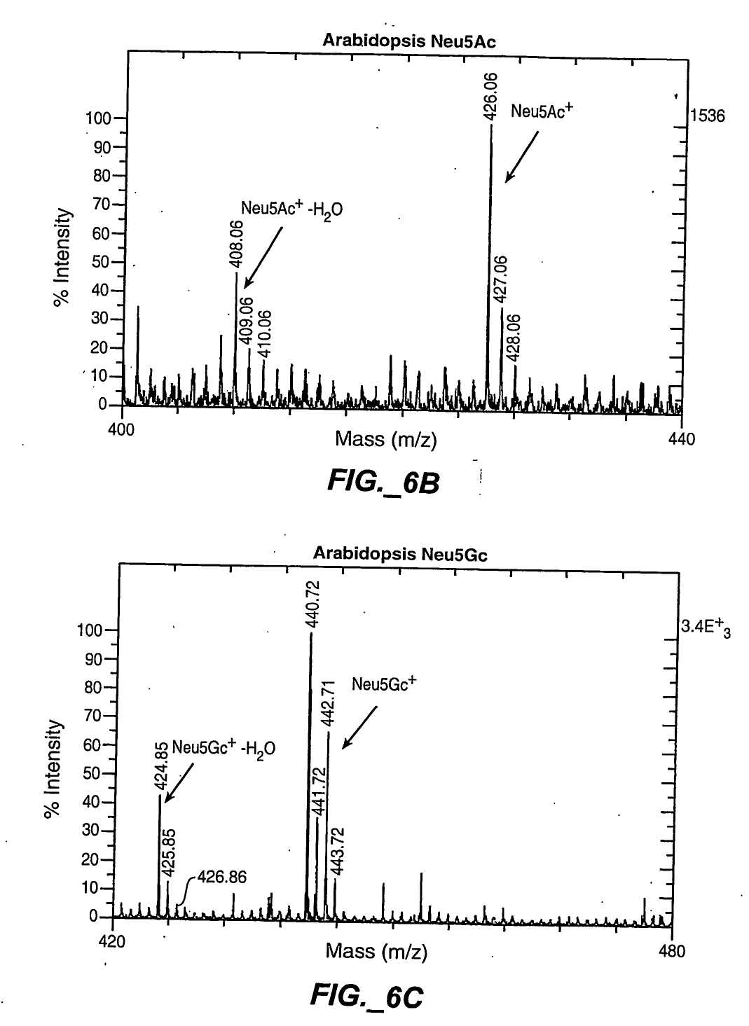

In FIG. 6, fractions collected from HPLC were concentrated and subjected to analysis using LC-ESI and MALDI-TOF. The DMB-labeled standards are shown in FIG. 6A. DMB-Neu5Ac m/z at 426 (FIG. 6B) and DMB-NeuGc m/z at 442 (FIG. 6B) confirm the presence of Neu5Ac and Neu5Gc respectively.

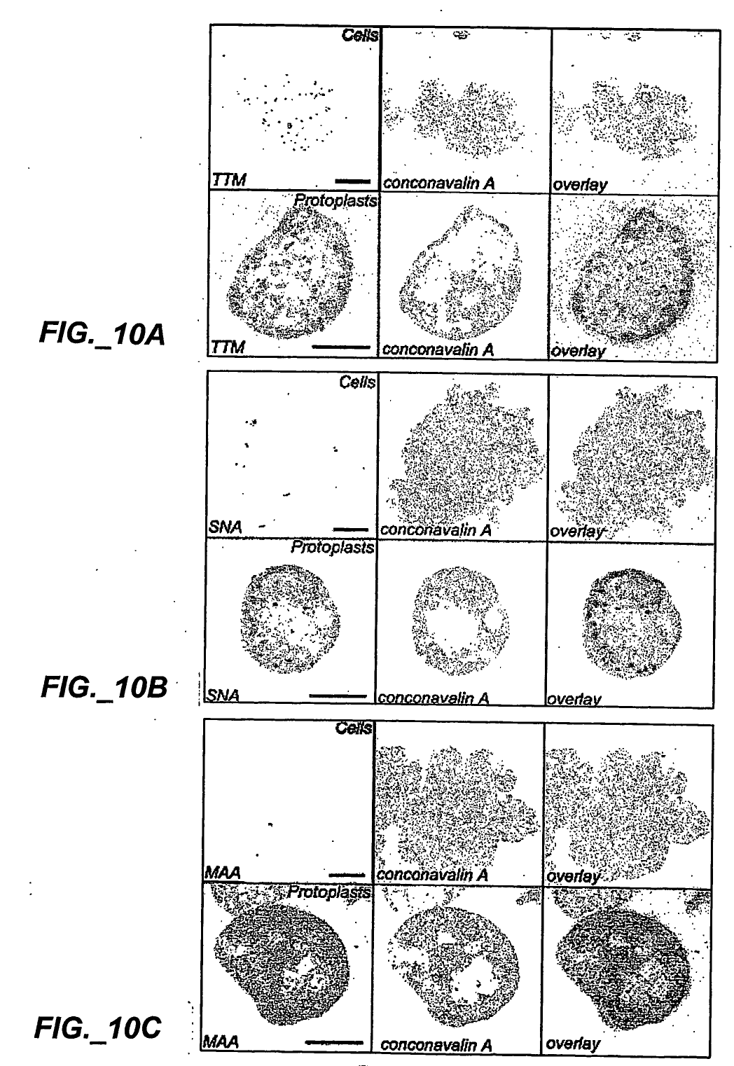

3. Detection of Sialylated Glycoconjugates by Immunofluorescence Microscopy as Shown in FIG. 10.

A. thaliana suspension-cultured cells (top panel) were fixed in 4% formaldehyde and probed with biotinylated lectins (SNA-I, MAA, TTM), specific for sialic acid to study cell surface expression of sialic acid. Secondary probe was anti-biotin antibody conjugated to Cy2 (λₑₓ 495 nm, λₑₘ 519 nm). To study intracellular distribution of sialoglycoconjgates, protoplasts (bottom panel) were prepared by digesting formaldehyde-fixed cells with cellulase and pectinase enzymes at 30° C. for 3 hours. Protoplasts were treated with 10% triton X-100 and probed with lectins as mentioned earlier. For all the experiments, ConA-Alexa568 (λₑₓ 579 nm, λₑₘ 603 nm) was used as a positive control and 1% BSA was used to inhibit non-specific binding. Sialic acid-specific lectins did not show binding to the cell surface. However, binding of these lectins was significant when protoplasts were prepared.

Sialylated glycoconjugates in suspension-cultured cells were detected using biotinylated SNA and MAA lectins. Anti-biotin antibodies conjugated to Cy2 were used to detect sialylated glycoconjugates using confocal microscopy. Cells were treated with pectinase and cellulase to detect intracellular sialoglycoconjugates or this treatment was omitted to detect cell-surface expression of sialoglycoconjugates. Plant cells were treated with BSA to prevent nonspecific binding and observed with a confocal microscope. These experiments suggest that SA motifs are primarily located on intracellular and/or cell membrane glycoconjugates, but not on the plant cell wall, as shown by the comparison of cells and protoplasts for TTM (FIG. 10A), SNA (FIG. 10B) and MAA (FIG. 10C).

4. Organ-Specific Distribution of Sialylated Glycoproteins in A. thaliana Plants.

A. thaliana plants were grown in a 16/8 light cycle at 23° C. After maturation, different organs of A. thaliana were collected and total protein extracts from stem, rosette leaves, reproductive leaves and flowers were prepared. Proteins were separated on SDS-PAGE and probed with SNA-I and MAA to study the distribution of sialylated glycoconjugates in different organs.

The preliminary results show that sialoglycoproteins are differentially distributed in all organs of the A. thaliana plant. Quantification of SAs by the ferri-corcinol method suggested approximately 2% sialylation of A. thaliana total proteins. Sialylation was also detected in cultured cells of other plant species, such as Nicotiana tabacum and Medicago sativa.

5. Data Mining.

Nucleotide and protein sequences encoding experimentally-authenticated UDP-GlcNAc-2-epimerase/ManNAc-6-kinases, SA synthases, CMP-SA transporters, and STs from Homo sapiens, Mus musculus, Rattus norvegicus, Drosophila melanogaster, and Sus scrofa were queried against A. thaliana database using BLASTN and BLASTP algorithms. In the case of UDP-GlcNAc-2-epimerase/ManNAc-6-kinase and SA synthase, no A. thaliana genes were identified that showed significant sequence homology (an expect value <1E-4). However, queries using CMP-SA transporters and STs (α-2,3-, α-2,6-, and α-2,8-) from each of the aforementioned organisms identified A. thaliana genes encoding proteins with significant homology (>1E-4). Two possible candidates (AT5G41760 and AT3G59360) for CMP-SA transporters were found: AT5G41760 shows the highest degree of homology to the Mus musculus CMP-SA transporter (1E-27), while AT3G59360 is homologous (1E-17) to the Homo sapiens CMP-SA transporter. Both proteins are predicted to have 10 membrane spanning regions based on Kyte and Doolittle hydrophobicity algorithms.

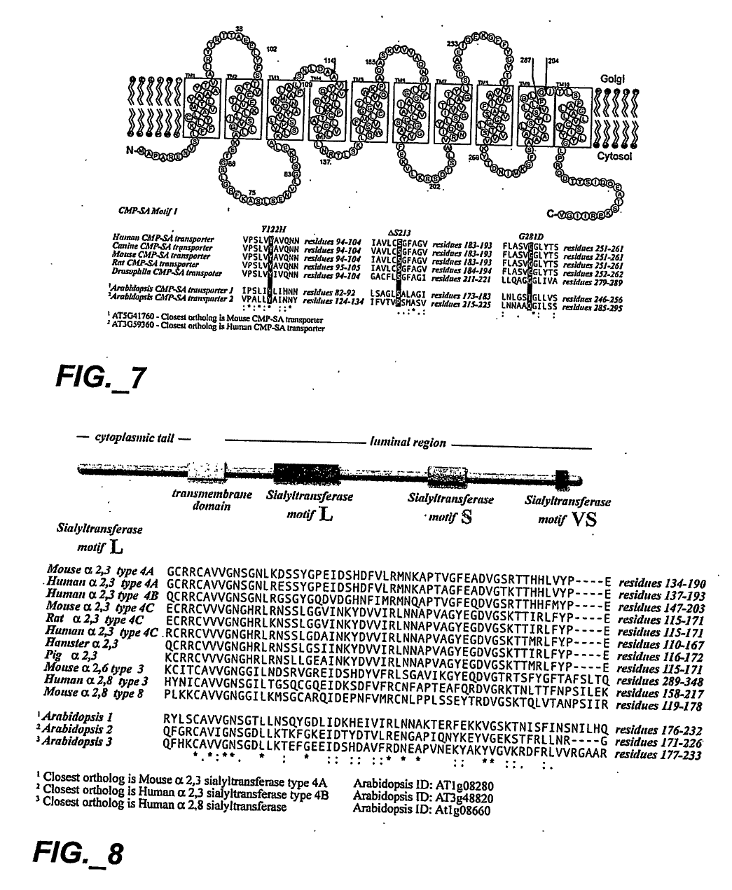

FIG. 7 shows the topology and amino acid sequence alignment of selected CMP-SA transporters. FIG. 7 shows an alignment of these A. thaliana candidates with other CMP-SA transporters in regions previously identified by Olemann et al. (2001) to be critical for CMP-SA transporter function. Also shown is a diagram generated by Eckhardt et al. (1999) illustrating experimentally-determined murine CMP-SA transporter topology that we have modified to show a relationship with the alignment below it.

Modifications in the Mus musculus CMP-SA transporter of Sα-213 and G281D ameliorated transporter activity while Y122H did not (Olemann et al., 2001). Surprisingly, both A. thaliana candidate genes have conserved tyrosine residues at a position correlating to Y122 in the mouse CMP-SA transporter, but only AT5G41760 has a conserved serine corresponding to the mouse S213. Neither candidate A. thaliana gene has the conserved glycine at position 281. The relevance of these residues as they relate to CMP-SA transporter function in A. thaliana can ultimately be determined experimentally.

BLAST analysis of STs from the organisms listed above identified three homologs in A. thaliana. AT1G08280 shows the highest degree of homology to mouse α-2,3 type 4A ST (2E-12), while the other two A. thaliana candidates AT3G48820 and AT1g08660 show greatest homologies to human α-2,3 type 4B ST (6E-14) and human α-2,8 ST, respectively (5E-11).

Depicted in FIG. 8 is a diagram of a typical ST with salient features noted. In most STs, the transmembrane domain and the three sialylmotifs, L, S and VS are conserved. FIG. 8 also shows an alignment of residues in sialylmotif L of mammalian and A. thaliana genes. The three putative STs from A. thaliana are highly conserved in the sialylmotif L.

Supplemental Methods for Determining Presence of SA.

1. Preparation of Cell Extract.

All experiments were carried out with 7-day old suspension cultured cells of Arabidopsis thaliana. Cells were washed with water and suspended in resuspension buffer (25 mM HEPES pH 7.5, 2 mM DTT, 0.5 mM PMSF and 1 μg/ml leupeptin). Total protein extract was prepared by subjecting to French-press (American Instrument Company, MO) at 10,000 psi. Membrane-bound proteins were extracted with 0.05% β-dodecyl maltoside. Samples were centrifuged at 5000 rpm for 20 minutes at 4° C., supernatant was collected and passed through PD10 columns (Amersham, NJ) for removal of salts and detergent. Eluant from PD10 column was used as total cell protein extract.

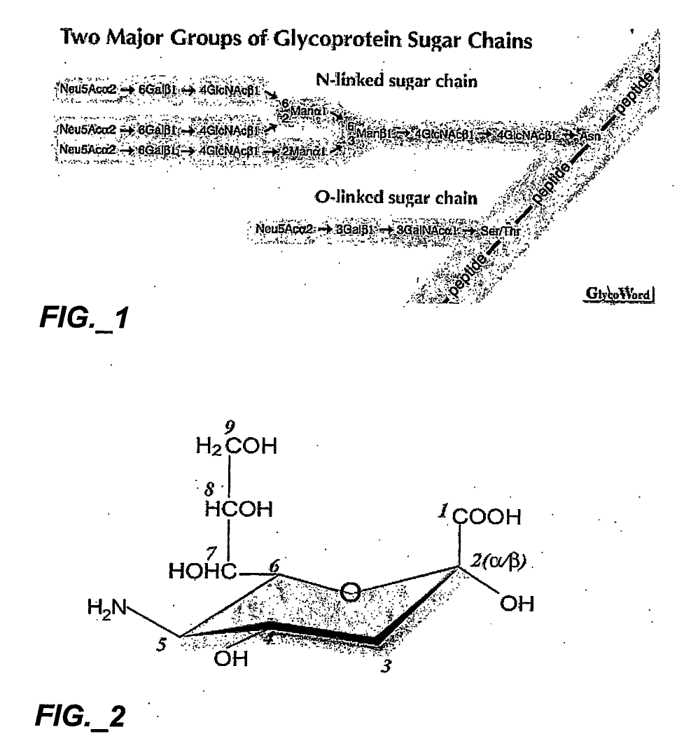

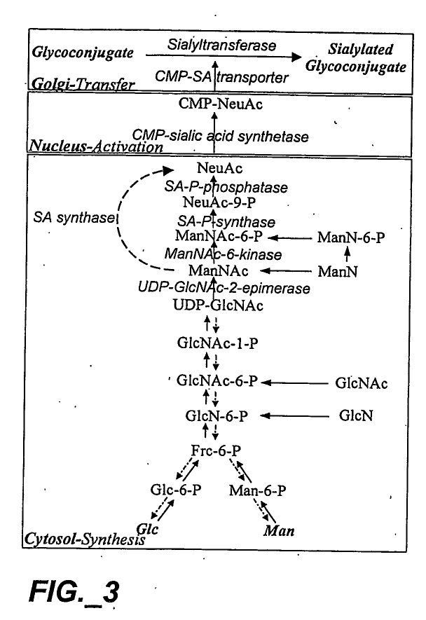

The total amount of protein was determined by Bradford protein quantification method (Bio-rad, CA) according to supplier's instructions. For sodium dodecyl sulfate-polyacrylamide gel electrophoresis (SDS-PAGE) analysis, 15 μg A. thaliana proteins were used (FIG. 1). 0.5 μg fetuin and asialofetuin were loaded on the SDS-PAGE as positive and negative controls, respectively (fetuin and asialofetuin can be detected at 100 ng levels by sialic acid specific lectins used for the analysis).

Lectin Blot and Lectin-Affinity Chromatography.

Proteins were separated on SDS-PAGE and electro-transferred onto Polyvinylidene fluoride (PVDF) membrane. Membranes were washed three times (10 min.×3) in Tris-Buffered Saline (TBS) (50 mM Tris-HCl, pH 7.5, 150 mM NaCl) and blocked in 1% gelatin containing 0.05% Tween 20™ for 3 hours at room temperature with gentle shaking. Membranes were then incubated in lectin incubation buffer (50 mM Tris-HCl, pH 7.5, 150 mM NaCl, 1 mM CaCl₂, 1 mM MgCl₂, 1 mM MnCl₂) containing biotinylated SNA-I (10 μg) and MAA (50 μg) lectins for 2 hr. Membranes were washed three times with TBS (10 min.×3) and incubated in streptavidin-conjugated alkaline phosphatase for 1 hour.

Membranes were washed three times (10 min.×3) and lectin binding to specific glycan residues was calorimetrically detected by 5-Bromo-4-chloro-3-indolyl phosphate/Nitro Blue Tetrazolium (Sigmafast™ BCIP/NBT, Sigma, MO). For lactose inhibition experiment, lectins were pre-incubated in 100 mM lactose solution. For this experiment, blocking buffer, lectin incubation buffer as well as the wash buffer contained 100 mM lactose.

Sepharose-conjugated SNA-I and MAA columns were equilibrated at room temperature with TBS (10 mM tris-HCl, pH 7.5, 150 mM NaCl, 1 mM CaCl₂, 1 mM MgCl₂, 1 mM MnCl₂). Total protein extract from A. thaliana was loaded on the lectin-affinity column, washed with TBS (at least five times the column volume) to remove unbound proteins. Sialic acid containing proteins were eluted using 100 mM lactose in TBS and 20 mM unbuffered ethylenediamine.

The column eluant was dialyzed and concentrated using Macrosep centrifugal devices (VWR, PA). The eluant was also subjected to digestion with α2-3,6 sialidase (Clostridium perfringens, Calbiochem, CA). Affinity-purified proteins that were sialidase treated and untreated, were separated on SDS-PAGE, transferred on to PVDF membrane and detected with SNA-I and MAA lectins.

Purification of Sialic Acids from A. thaliana Cells.

A. thaliana cell extract was prepared as described above and proteins were precipitated using ammonium sulfate (72%) to eliminate the possibility of contamination with KDO. Precipitated proteins were dialyzed against water overnight at 4° C. by change of water at least three times using spectrapore dialysis membrane (MWCO: 2000 Da, Pierce, Ill.) and then used for release of sialic acids. Sialic acid residues were released from dialyzed proteins by subjecting to mild acid hydrolysis (2M acetic acid, 80° C., 3 hour) in presence of 1% Butylated Hydroxytoluene (BHT). Acid-hydrolyzed protein samples were incubated on ice for 30 minutes.

Released sialic acids were collected by dialyzing overnight against H₂O using spectrapore dialysis membrane (MWCO: 1000 Da, Pierce, Ill.) at 4° C. Diffusate from the membrane was collected, lyophilized and subsequently resuspended in water. Sialic acid samples were loaded on Dowex AG-50WX2 (Hydrogen) column (Pierce, Ill.) and washed with water. Eluant was collected and pH was adjusted with 10 mM sodium formate pH 5.5 buffer. Eluant was then subjected to Dowex AG50×8 (formate) column (Bio-Rad, CA). The column was washed using 10 mM formic acid. Sialic acids were eluted using 1 M formic acid. Eluant was lyophilized, reconstituted in water, labeled with 1,2-diamino-4,5-methyleneoxybenzene (DMB, a fluorescent dye specific for sialic acids) and analyzed on RP-HPLC (as shown in FIG. 2) or mass spectrometry.

Structural Analysis of Sialic Acids.

DMB-SA derivatives were separated on a reversed-phase C18 column (Phenomenex, CA) on an Agilent 1100 workstation under isocratic conditions of 9% acetonitrile, 7% methanol and 84% water at flow rate of 0.65 ml/min. As shown in FIG. 4A, commercially available Neu5Ac and Neu5Gc were used as standard SAs. In A. thaliana samples, a prominent peak corresponding to Neu5Gc and a smaller peak corresponding to Neu5Ac were detected, indicating the presence of these two SAs on A. thaliana glycoconjugates.

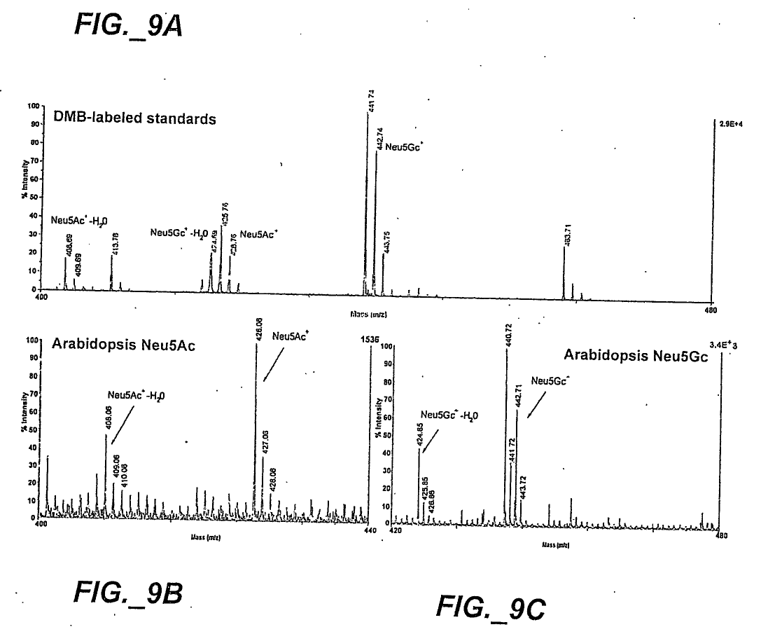

Results similar to FIG. 4 were observed when sialic acids were removed from glycoconjugates by α2-3,6 sialidase treatment. HPLC-purified DMB-SA derivatives were used for further analysis, using MALDI-TOF (Voyager DE-STR, Applied Biosystems). As shown in FIG. 9, the parent ion masses of sialic acids purified from A. thaliana were confirmed by comparing them with commercially available standard Neu5Gc and Neu5Ac (FIG. 9A). The m/z at 426 confirms the presence of Neu5Ac (FIG. 9B) and m/z at 442 confirms the presence of Neu5Gc (FIG. 9C).

Methods for Engineering Plant Pathways

These genomic and experimental data clearly show that plants contain genes involved in sialylation and produce sialylated glycoconjugates. However, none of the biosynthetic and regulatory steps of the SA biosynthesis pathway are known in plants. This detailed description further discloses specific metabolic engineering approaches that will assist in the understanding and establishment of the molecular and biochemical mechanisms of SA biosynthesis pathway in plants.

The presence of sialylated glycoproteins in A. thaliana raises important questions about SA synthesis, transport and transfer, for which the scientific community still has no experimental knowledge. Fortunately, there are extensive studies on SA biosynthesis in animals and bacteria that clearly show that these organisms utilize similar metabolic pathways and that the enzymes that catalyze identical chemical reactions share relatively high homology on amino acid level. This evolutionary conservatism defines the identification of genes/proteins by comparative genomic/proteomic database analyses as the first logical step in elucidating the biosynthesis of SA in plant cells.

As described in our preliminary studies, in animals, UDP-GlcNAc-2-epimerase/ManNAc-6-kinase and NeuAc phosphate synthase play crucial roles in SA synthesis. The epimerase domain of the mammalian UDP-GlcNAc-2-epimerase/ManNAc-6-kinase shares high level of sequence homology to bacterial UDP-GlcNAc-2 epimerase, as do the bacterial SA synthase and mammalian NeuAc phosphate synthase genes.

The absence of plant homologs to mammalian UDP-GlcNAc-2-epimerase/ManNAc-6-kinase and NeuAc phosphate synthase (bacterial SA synthase), requires further examination of whether these enzyme activities exist in the plant cells, as the possibility that different protein entities might have the same or similar functional characteristics could not be ruled out.

Overexpression of human UDP-GlcNAc-2-epimerase/ManNAc-6-kinase and NeuAc phosphate synthase genes in A. thaliana followed by measurement of the amount of SA and levels of sialylation compared to untransformed plants, would support or overrule the involvement of these enzymes and their reaction products in plant SA biosynthesis pathway. The human genes encoding UDP-GlcNAc-2-epimerase/ManNAc-6-kinase and NeuAc phosphate synthase will be received from our collaborators, Prof. Werner Reutter (Freie University, Berlin, Germany) and Prof. Michael Betenbaugh (Johns Hopkins University, MD), respectively.

Transport of CMP-SA to Golgi lumen and the subsequent transfer of SAs to the non-reducing termini of the oligosaccharide chains of glycoconjugates are conserved processes among animals, insects and plants. Several putative CMP-SA transporter and ST genes have been identified in A. thaliana database by using their mammalian and Drososphila counterparts as queries. Plant CMP-SA transporters and STs can be functionally characterized by transgenic approach, using both, gene over-expression and down-regulation.

The results from the impact of these manipulations on the amount of SA and sialylation levels provide an evaluation of gene product function, and enable identification of A. thaliana sialylated glycoproteins, further elucidating the biological significance of their terminal SA residues. For down-regulation experiments, collections of targeted and random T-DNA mutants from various sources can be used.

We have identified multiple T-DNA mutants for each of the A. thaliana genes described above (in many cases multiple T-DNA insertions at different gene positions are available for the same gene). The presence and location of the T-DNA can be verified by PCR or hybridization techniques, and the plants can then be analyzed for alterations in the levels of sialylated proteins and free SA.

Because functional redundancy may, at times, result in the effect of T-DNA insertional inactivation being somewhat unclear, the implementation of RNAi technology is used as a complementary approach. A. thaliana STs will also be overexpressed in E. coli, to ensure quick production of enough amount of functionally active proteins that will be used for further biochemical characterization.

The experimental protocols and assays described below are non-limiting examples for practicing the methods described herein for different tissues of A. thaliana plants.

Vector Construction for Plant Transformation.

1. Construction of Plant Binary Vectors for Agrobacterium-Mediated Transformation.

A GATEWAY cloning technology (Invitrogen) that exploits site-specific recombination system of bacteriophage λ of E. coli can be used for construction of plant expression vectors. The cDNAs—fragments (for hp RNA constructs) or whole length, can be PCR-amplified (Pfu Turbo DNA polymerase, Stratagene), in the latter case with (because of the lack of antibodies specific for sialyltransferases, CMP synthetase, CMP transporter and UDP-GlcNAc-2-epimerase/ManNAc-6-kinase), or without a myc epitope appended to the N-terminus. A directional Topo®-clonning strategy is implemented to position them between the attL sites of pENTR/SD/D-Topo vector (Invitrogen).

By the means of LR reaction (Invitrogen), the target genes can be transferred to GATEWAY-compatible binary vectors under the control of CaMV ³⁵S promoter and nos terminator. A pKZ7WG2 and pK7GWIWG2 is used for over-expression and co-suppression of the target genes, respectively. Both vectors are built within the pPZP200 backbone and contain streptomycin/spectinomycin resistance gene for bacterial selection and kanamycin resistance plant selectable marker gene (Karimi et al., 2002). Between the inverted GATEWAY fragments pK7GWIWG2 contains Arabidopsis intron (ac007123.em_pl) with ideal features for efficient splicing (A+T-rich with a branch site close to the consensus). In plants, it produces double-stranded RNA (hairpin RNA) from the inserted sequence of interest, thus triggering in an efficient way post-transcriptional gene silencing.

2. Construction of Expression Vectors for E. coli Transformation.

The cDNAs can be amplified by PCR (Pfu Turbo DNA polymerase, Stratagene) with appropriate linker sequences (if necessary) and cloned into the multiple cloning site of pET14b (Novagen) by standard restriction digestion and ligation procedures. pET14b is a bacterial expression vector employing a T7 promoter/terminator and an in-frame N-terminal poly histidine (6×His) affinity tag that yields high levels of expression in E. coli after IPTG induction and allows easy detection and purification of the recombinant protein.

Transformation of Arabidopsis thaliana.

1. Agrobacterium-Mediated Transformation of Arabidopsis Plants.

Stable Agrobacterium-mediated transformation in planta is carried out using vacuum-infiltration procedure as described by Clough and Bent (1998). After 24 hours co-cultivation, the plants are rinsed with water and grown to maturity. Seeds are collected, surface sterilized and grown on selection medium (0.5×MSO containing 0.7% (w/v) agar, supplemented with 25-50 microgram/ml kanamycin). The transformants are transferred to soil and their transgenic status confirmed by molecular analyses.

2. Biolistic Transformation of Suspension Culture Cells (for Subcellular Localization).

Cells from four day post-subcultured Arabidopsis suspension cultures are collected by centrifugation, resuspended in an equal volume of transformation medium-TM (growth medium without kinetin and NAA, supplemented with 250 mM sorbitol and 250 mM mannitol), spread on filter papers and allowed to equilibrate for 1 hours at RT. A microprojectile bombardment (PDS 1000/He Bio-Rad) with plasmid DNA precipitated onto M-17 tungsten particles will be carried out as described by Banjoko and Trelease (1995). Cells are left in darkness for 2, 5, 10, or 20 hours to undergo post-bombardment transient gene expression before the analysis.

3. Immunofluorescence Microscopy.

Bombarded cells are fixed in 4% (w/v) formaldehyde in 0.5×Arabidopsis TM for 1 h at RT, washed three times in phosphate buffered saline (PBS, 4.3 mM Na₂HPO₄, 1.4 mM KH₂PO₄, 2.7 mM KCl, 137 mM NaCl, pH 7.4) and incubated in 0.1% (w/v) Pectolyase Y-23 and 0.1% (w/v) Cellulase-RS (Karlan Research Products, Santa Rosa, CA) for 2 hours at 30° C. During incubation plasma and organell membranes are permeabilized by treatment with 0.3% (v/v) Triton X-100 for 15 min at RT. Fixed and permeabilized cells are processed for indirect immunofluorescence as described previously (Flynn et al., 1998). Primary and dye-conjugated secondary antibodies are diluted as necessary.

4. DNA and RNA Analyses.

Isolation of plasmid and genomic DNA, RNA isolation, Southern and Northern blot analyses, PCR and RT-PCR are carried out according to standard protocols (Ausubel et al., 2001).

5. SDS PAGE, Western and Lectin Blot Analyses.

Proteins are prepared and separated on 4-20% Ready Gel® Precast polyacrylamide gels (Bio-Rad). For Western analyses, proteins are transferred to PVDF membranes in a semi-dry transfer apparatus (Bio-Rad) employing an anode (0.3 M Tris, 0.1 M glycine, 0.0375% SDS)/cathode (0.3 M aminocaproic acid, 0.03 M Tris, 0.0375% SDS) buffering system. The blots are blocked with 5% (w/v) instant milk (Carnation Brand) in Tris-buffered saline for 1 hour.

The target proteins are detected using specific primary antibodies and alkaline phosphatase-coupled secondary antibodies diluted (1:10,000) in TBST containing 1% (w/v) dried milk. Immunoblots are washed three times for 10 min with TBST before and after antibody incubations. Lectin analyses are carried out by Roche Molecular Biochemicals protocol. The blots are imaged using Immunostar substrate (Bio-Rad) and autoradiography film.

6. Purification of Recombinant Proteins Expressed in E. coli.

A fermentor-based-cell production of the target proteins, expressed in E coli, is performed as described by Panitch et al. (1997). Preparation of the protein samples and purification of His-tagged proteins of interest by metal chelation affinity chromatography follow the Novagen protocols. If the recombinant proteins require additional purification, additional chromatography methods are employed.

7. Determination of Free and Bound Sialic Acids.

Colorimetric assays adapted to the microscale and HPAEC (High pH Anion Exchange Chromatographic) are used to detect and quantify SAs. Bound SAs are the estimated by the periodate-resorcinol microtiter plate assay capable of detecting nano mole amounts (Bhavanandan and Sheykhnazari, 1993). The advantage of this approach is that there is no need for pretreatment and even crude samples can be analyzed. Free SAs are estimated by the micro scale version of the Warren thiobarbituric acid method (Yeh et al., 1971).

This widely used colorimetric assay was believed to be specific for free SA; however, recently it was shown that the reagent also reacts with SA linked 2, 6 to unsubstituted GalNAc (Bhavanandan et al., 1998). Further, compounds that are present in plants such as KDO (3-deoxy-D-manno-2-octulosonic acid) and KDN (2-keto-3-deoxy-D-glycero-D-galacto-nonic acid) are also known to give positive signals in the periodate-thiobarbituric acid assay (Kitajima et al., 1992). Therefore, it is essential to confirm that a positive reaction in this assay is indeed due to free SAs by additional techniques.

Thus, all samples are also analyzed by HPAEC. Using this technique we were able to detect pico mole quantities of SA and also distinguishes between various forms of SA. Samples containing bound SA are first treated with mild acid (0.1 N sulfuric acid, 80 to 60 min) to hydrolyze the ketosidic bond. The samples containing free SA and the hydrolysates are analyzed on a CarbPac PA-10 column (Dionex Corp.) and using pulsed amperometric detection (Manzi et al., 1990; Rohrer, 2000).

If any unusual SA forms are detected by HPAEC in the plant samples, these are isolated by RP-HPLC, derivatized with DMB and analyzed by LC-ESI and MALDI-TOF mass spectrometry as described above.

8. Enzymatic Assays.

The following enzymatic assays are then carried out with the appropriate modifications required for plant sample analyses.

A. UDP-GlcNAc 2-Epimerase and ManNAc-6-Kinase.

UDP-GlcNAc 2-epimerase and ManNAc-6-kinase activity is measured by a method described by Effertz et al (1999). In brief, known quantities of total soluble proteins from the plant cell extracts are incubated with the reaction mixtures for UDP-15. GlcNAc 2-epimerase and ManNAc-6-kinase. For UDP-GlcNAc-2-epimerase assay, the mixture includes: 45 mM Na₂HPO₄, pH 7.5, 10 mM MgCl₂, 1 mM UDP-GlcNAc, 25 nCi of UDP-[14C]GlcNAc. For the ManNAc kinase assay the mixture contains: 60 mM Tris/HCl, pH 8.1, 20 mM MgCl₂, 5 mM ManNAc, 50 nCi of [14C]ManNAc, 20 mM ATP (disodium salt).

After incubation for pre-determined time, the reaction is stopped by the addition of ice-cold ethanol. Radiolabeled products are separated by paper chromatography, eluted from the paper and estimated by liquid scintillation counting. One unit of enzyme activity is defined as that catalyzing the formation of 1 mmol of product/min at 37° C. Specific activity is expressed as milliunits/mg of protein.

B. SA Synthase.

In vitro activity assay is carried out according to Lawrence et al (2000). 5 μl of substrate solution is incubated with 20 μl of cell lysate (30 or 60 min) at 37° C. The substrate solution contains 10 mM MnCl₂, 20 mM PEP, and either 5 mM ManNAc, 5 mM ManNAc-6-P or 25 mM mannose 6-phosphate. Assays performed on samples containing boiled lysates can be used as controls.

Following incubation, all samples are boiled for 3 min, centrifuged for 10 min at 12,000×g, and split into two 10-ml aliquots. One aliquot is treated with 9 units of calf intestine alkaline phosphatase (Roche Molecular Biochemicals) along with 3 ml of accompanying buffer, while the other aliquot is diluted with water and buffer. Alkaline phosphatase (AP)-treated aliquots are incubated for 4 hours at 37° C., and 10 μl of both AP-treated and -untreated samples are reacted with DMB as described above.

C. CMP-Sialic Acid Transporter.

The in vitro transport assay is performed essentially as described previously (Aoki et al., 2001) with slight modifications. The reaction is started by addition of the plant cell membrane preparation to the reaction mixture (0.8 M sorbitol, 10 mM Tris-HCl (pH 7.0), 1 mM MgCl₂, 0.5 mM dimercaptopropanol, and ³H-labeled nucleotide sugar-CMP-3H SA. The reaction is stopped using ice-cold stop buffer (0.8 M sorbitol, 10 mM Tris-HCl (pH 7.0), and 1 mM MgCl₂), and poured onto a nitrocellulose filter. The filter is washed three times with stop buffer and dried. After solubilization, the radioactive material on the filters is estimated liquid scintillation counting.

D. Sialyltransferase (ST).

ST activity is assayed according to Carey and Hirschberg (1980). In brief, membrane fraction of the plant cells prepared by sucrose step gradient centrifugation is incubated with the reaction mixture. A typical reaction mixture contains CMP-3H-sialic acid [0.3 mCi], asialofetuin (and other suitable acceptors), and buffer [33 mM Na₃PO₄, 100 mM NaCl, pH 7.5, and 0.2% Triton X-100]. Controls include reaction mixtures minus cell fractions or asialofetuin. Fetuin has both N-linked and O-linked saccharides and therefore, asialofetuin is a good initial substrate to measure total activity of sialyltransferase(S).

Subsequently, specific asialosubstrates, such those derived from alpha-1-acid glycoproteins or mucins, are tested to distinguish the substrate specificity of plant sialyltransferases. The reaction is stopped by addition of cold 1% phosphotungstic acid (PTA) in 0.5 N HCl. The precipitated glycoproteins are pelleted by centrifugation and rinsed three times with fresh 1% PTA/0.5 N HCl. The pellet is dissolved in 1 N NaOH transferred to a scintillation vial and neutralized with 4 N HCl. The product radioactivity is estimated by liquid scintillation counting.

E. SALyase.

SA lyase is partially purified from extracts of A. thaliana by ammonium sulfate precipitation method. Activity of lyase is measured by adding known amount of exogenous SA at 37° C. for 15 minutes. The reaction is stopped by heating the reaction mixture at 100° C. Production of pyruvate (degradation product of SA) is measured by adding lactate dehydrogenase and NADH. As pyruvate is converted to lactate, NADH is oxidized to NAD. Oxidation of NADH (production of pyruvate) can be estimated by measuring the change of the absorbance at 340 nm. One unit of enzyme is defined as the quantity that yields 1.0 μmole of pyruvate in above-described conditions.

Various embodiments of the invention are described above in the Drawings and Description of Various Embodiments. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventor that the words and phrases in the specification and claims be given the ordinary and accustomed meanings to those of ordinary skill in the applicable art(s). The foregoing description of a preferred embodiment and best mode of the invention known to the applicant at the time of filing the application has been presented and is intended for the purposes of illustration and description. It is not intended to be exhaustive or to limit the invention to the precise form disclosed, and many modifications and variations are possible in the light of the above teachings. The embodiment was chosen and described in order to best explain the principles of the invention and its practical application and to enable others skilled in the art to best utilize the invention in various embodiments and with various modifications as are suited to the particular use contemplated. Therefore, it is intended that the invention not be limited to the particular embodiments disclosed for carrying out this invention, but that the invention will include all embodiments falling within the scope of the appended claims

### REFERENCES

1. Aoki K, Ishida N, Kawakita M. Substrate recognition by UDP-galactose and CMP-sialic acid transporters. Different sets of transmembrane helices are utilized for the specific recognition of UDP-galactose and CMP-sialic acid. Journal of Biological Chemistry 276: 21555-61 (2001).
2. Ashwell G, Morell AG. The role of surface carbohydrates in the hepatic recognition and transport circulating glycoproteins. Advances in Enzymology 41: 99-128 (1974).
3. Ausubel FM. Current protocols in molecular biology. Ausubel F M, Brent R, Kingston R E, Moore D D, Seidman J G, Smith J A, Struhl K. John Wiley & Sons (2001).
4. Banjoko A, Trelease R N. Development and application of an in vivo plant peroxisome import system. Plant Physiology 107(4):1201-8 (1995).
5. Bhavanandan V P, Sheykhnazari M. Adaptation of the periodate-resorcinol method for determination of sialic acids to a microassay using microtiter plate reader. Analytical Biochemistry 213: 438-40 (1993).
6. Bhavanandan V P, Ringler N J, Gowda D C. Identification of the glycosidically bound sialic acid in mucin glycoproteins that reacts as “free sialic acid” in the Warren assay. Glycobiology 8(11):1077-86 (1998).
7. Carey D J, Hirschberg C B. Kinetics of glycosylation and intracellular transport of sialoglycoproteins in mouse liver. Journal of Biological Chemistry 225: 4348-4354 (1980).
8. Clough S J, Bent A F. Floral dip: a simplified method for Agrobacterium-mediated transformation of Arabidopsis thaliana. Plant Journal 16(6):735-43 (1998).
9. Comb D B, Roseman S. The sialic acids. I. The structure and enzymatic synthesis of N-acetylneuraminic acid. Journal of Biological Chemistry 235: 2529-2537 (1960).
10. Corfield A P, Schauer R, Wember M. The preparation of CMP-sialic acids by using CMP-acylneuraminate synthase from frog liver immobilized on sepharose 4B. Biochemistry Journal. 177(1):1-7 (1979)
11. Datta A K, Sinha A, Paulson J C. Mutation of the sialyltransferase S-sialylmotif alters the kinetics of the donor and acceptor substrates. Journal of Biological Chemistry 273: 9608-9618 (1998).
12. Eckhardt M, Gotza B, Gerardy-Schahn R. Membrane Topology of the Mammalian CMP-Sialic Acid Transporter. Journal of Biological Chemistry 274: 8779-8787 (1999).
13. Effertz K, Hinderlich S, Reutter W. Selective Loss of either the Epimerase or Kinase Activity of UDP-N-acetylglucosamine 2-Epimerase/N-Acetylmannosamine Kinase due to Site-directed mutagenesis Based on Sequence Alignments. Journal of Biological Chemistry 274(40):28771-8 (1999).
14. Flynn C R, Mullen R T, Trelease R N. Mutational analyses of a type 2 peroxisomal targeting signal that is capable of directing oligomeric protein import into tobacco BY-2 glyoxysomes. Plant Journal 16(6):709-20 (1998).
15. Gerardy-Schahn R, Oelmann S, Bakker H. Nucleotide sugar transporters: biological and functional aspects. Biochimie 83: 775-82 (2001).
16. Geremia R A, Harduin-Lepers A, Delannoy P. Identification of two novel conserved amino acid residues in eukaryotic sialyltransferases: implications for their mechanism of action. Glycobiology 7(2):5-7 (1997).
17. Gollub M, Schauer R, Shaw L. Cytidine monophosphate-Nacetylneuraminate hydroxylase in the starfish Asterias rubens and other echinoderms. Comparative Biochemistry and Physiology Part B 120: 605-15 (1998).
18. Hara S, Takemori Y, Yamaguchi M, Nakamura M, Ohlkura Y. Fluorometric high-performance liquid chromatography of N-acetyl- and N-glycolelneuraminic acids and its application to their mcrodetermination in human and animal sera, glycoproteins and glycolipids. Analytical Biochemistry 164: 138-145 (1987).
19. Hinderlich S, Stasche R, Zeitler R, Reutter W. A bifunctional enzyme catalyzes the first two steps in N-acetylneuraminic acid biosynthesis of rat liver. Purification and characterization of UDP-N-acetylglucosamine 2-epimerase/N-acetylmannosamine kinase. Journal of Biological Chemistry 272(39):24313-8 (1997).
20. Hong Y, Stanley P. Lec3 Chinese hamster ovary mutants lack UDP-N-acetylglucosamine 2-epimerase activity because of mutations in the epimerase domain of the Gne gene. Journal of Biological Chemistry 278(52):53045-54 (2003).
21. Inoue Y, Inoue S. Diversity in sialic and polysialic acid residues and related enzymes. Pure and Applied Chemistry 71: 789-800 (1999).
22. Joshi L, Shuler M L, Wood H A. Production of a sialylated N-linked glycoprotein in insect cells. Biotechnology Progress 17(5):822-7 (2001)
23. Karimi M, Inze D, Depicker A. GATEWAY vectors for Agrobacterium-mediated plant transformation. Trends in Plant Sciences 7(5):193-5 (2002).
24. Kelm S, Schauer R. Sialic acids in molecular and cellular interactions. In: Jeon K W, Jarvik J W (eds) International Review of Cytology, pp. 137-40. Academic Press, San Diego (1997).
25. Keppler O T, Hinderlich S, Langner J, Schwartz-Albeiz R, Reutter W, Pawlita M. UDP-GlcNAc 2-epimerase: A regulator of cell surface sialylation. Science 284: 13721376(1999).
26. Kim K, Lawrence S M, Park J, Pitts L, Vann W F, Betenbaugh M J, Palter K B. Expression of a functional Drosophila melanogaster N-acetylneuraminic acid (Neu5Ac) phosphate synthase gene: evidence for endogenous sialic acid biosynthetic ability in insects. Glycobiology 12: 73-83 (2002).
27. Kitajima K, Inoue S, Kitazume S, Inoue Y. Analytical methods for identifying and quantitating deamidated sialic acid (2-keto-3-deoxy-D-glycero-D-galactonononic acid) and alpha 2,8-linked poly(oligo)nonulosonate residues in glycoconjugates. Analytical Biochemistry 205(2):244-50 (1992).
28. Lawrence S M, Huddleston K A, Pitts L R, Nguyen N, Lee Y C, Vann W F, Coleman T A, Betenbaugh M J. Cloning and expression of the human N-acetylneuraminic acid phosphate synthase gene with 2-keto-3-deoxy-D-glycero-D-galacto-nononic acid biosynthetic ability. Journal of Biological Chemistry 275: 17869-77 (2000).
29. Manzi A E, Diaz S, Varki A. High-pressure liquid chromatography of sialic acids on a pellicular resin anion-exchange column with pulsed amperometric detection: a comparison with six other systems. Analytical Biochemistry 188(1):20-32 (1990).
30. Oelmann S, Stanley P, Gerardy-Schahn R. Point mutations identified in Lec8 Chinese hamster ovary glycosylation mutants that inactivate both the UDP-galactose and CMP-sialic acid transporters. Journal of Biological Chemistry 276: 26291-300 (2001).
31. Oetke C, Hinderlich S, Reutter W, Pawlita M. Epigenetically mediated loss of UDP-GlcNAc 2-epimerase/ManNAc kinase expression in hyposialylated cell lines. Biochemical and Biophysical Research Communication 308: 892-98 (2003).
32. Panitch, A, Matsuki, K, Cantor, E J, Cooper, S J, Atkins, EDT, Fournier, M J, Mason, T L, Tirrell, D A. “Poly(L-alanylglycine): multigram-scale biosynthesis, crystallization, and structural analysis of chain-folded lamellae” Macromolecules 30(1): 42-49 (1997).
33. Paulson J C, Colley K J. Glycosyltransferases. Structure, localization, and control of cell type-specific glycosylation. Journal of Biological Chemistry 264(30):17615-8 (1989).
34. Reutter W, Stasche R, Stehling P, Baum O. The biology of sialic acids: insight into their structure, metabolism and function in particular during viral infection. Glycosciences-Status and Perspectives. H-J Gabius and S Gabius (eds). pp. 245-259. Chapman Hall, Weinheim, Germany (1997)
35. Ringenberg M A, Steenbergen S M, Vimr E R. The first committed step in the biosynthesis of sialic acid by Escherichia coli K1 does not involve a phosphorylated N-acetylmannosamine intermediate. Molecular Microbiology 50: 961-75 (2003).
36. Rohrer J S. Analyzing sialic acids using high-performance anion-exchange chromatography with pulsed amperometric detection. Analytical Biochemistry 15;283(1):3-9 (2000).
37. Schauer R. Sialic acids and their roles as biological masks. Trends in Biochemical Sciences 10: 357-60 (1985).
38. Schauer R. Achievements and challenges of sialic acid research. Glycoconjugate journal 17: 485-499 (2000).
39. Shah M, Fujiyama K, Flynn C R, Joshi L. Sialylated endogenous glycoconjugates in plant cells. Nature Biotechnology 21: 1470-1471 (2003).
40. Shaw L, Schauer R. The biosynthesis of N-glycoloylneuraminic acid ccurs by hydroxylation of the CMP-glycoside of N-acetylneuraminic acid. Biological Chemistry Hoppe-Seyler 369: 477-86 (1988).
41. Shibuya N, Goldstein I J, Broekaert W F, Nsimba-Lubaki M, Peeters B, Peumans W J. The elderberry (Sambucus nigra L.) bark lectin recognizes the Neu5Ac(alpha 2-6)Gal/GalNAc sequence. Journal of Biochemistry 262: 1596-601 (1987).
42. Taniguchi N, Honke K, Fukuda M. Handbook of glycosyltransferases and related genes. Springer-Verlag, Tokyo (2002).
43. Tsuji S, Datta A K, Paulson J C. Systematic nomenclature for sialyltransferases. Glycobiology 6(7):5-7 (1996).
44. Vann W F, Tavarez J J, Crowley J, Vimr E, Silver R P. Purification and characterization of the Escherichia coli K1 neuB gene product N-acetylneuraminic acid synthetase. Glycobiology 7: 697-701(1997).
45. Varki A: Diversity in the sialic acids. glycobiology 2: 25-40 (1992).
46. Varki A. N-glycans. In: Essentials of Glycobiology. Ajit Varki R C, Jeffery Esko, Hudson Freeze, Gerald Hart, Jamey Marth (eds). pp. 85-100. Cold spring harbor laboratory press, New York (1999a).
47. Varki A. O-glycans. In: Essentials of Glycobiology. Ajit Varki R C, Jeffery Esko, Hudson Freeze, Gerald Hart, Jamey Marth (eds), pp. 101-113. Cold spring harbor laboratory press, New York (1999b).
48. Wang W C, Cummings R D. The immobilized leukoagglutinin from the seeds of Maackia amurensis binds with high affinity to complex-type Asn-linked oligosaccharides containing terminal sialic acid-linked alpha-2,3 to penultimate galactose residues. Journal of Biological chemistry 263: 4576-85 (1988).
49. Yeh A K, Tulsiani D R, Carubelli R. Neuramimidase activity in human leukocytes. Journal of Laboratory and Clinical Medicine 78(5):771-8 (1971).

1-47. (canceled)

48. A method for producing recombinant sialylated glycoproteins in plants, comprising administering a vector comprising a nucleic acid encoding the glycoprotein to a plant cell, wherein the plant cell expresses a plant CMP-sialic acid transporter and a plant sialyltransferase.

49. The method of claim 48, wherein the plant cell is a Arabidopsis thaliana, Nicotiana tabacum, or Medicago stativa plant cell.

50. The method of claim 48, wherein the plant cell comprises an expression vector encoding plant CMP-sialic acid transporter or plant sialyltransferase.

51. The method of claim 48, wherein the CMP-sialic acid transporter is encoded by a nucleic acid comprising a gene designated as AT5G41760 or AT3G59360.

52. The method of claim 48, wherein the plant sialyltransferase is encoded by a nucleic acid comprising a gene designated as AT1G08280, AT3G48820, or AT1g08660 gene.

53. A method for engineering plants to produce recombinant sialylated glycoproteins, comprising administering to the plant cell a vector comprising a nucleic acid encoding plant CMP-sialic acid transporter and plant sialyltransferase.

54. The method of claim 53, wherein the plant cell is a Arabidopsis thaliana, Nicotiana tabacum, or Medicago stativa plant cell.

55. The method of claim 53, wherein the CMP-sialic acid transporter is encoded by a nucleic acid comprising a gene designated as AT5G41760 or AT3G59360.

56. The method of claim 53, wherein the plant sialyltransferase is encoded by a nucleic acid comprising a gene designated as AT1G08280, AT3G48820, or AT1g08660 gene.

57. The method of claim 53, further comprising administering to the plant cell a vector comprising a nucleic acid encoding human UDP-GlcNAc-2-epimerase, ManNAc-6-kinase and NeuAC phosphate synthase.
